(19) United States
(12) Patent Application Publication
(10) Pub. No.: US 2016/0038214 A1
(43) Pub. Date: Feb. 11, 2016

(54) ELECTRIC-WARMING MOXIBUSTION DEVICE

(71) Applicant: YING SHIH WANG, Taipei (CN)

(72) Inventor: YING SHIH WANG, Taipei (CN)

(21) Appl. No.: 14/455,948

(22) Filed: Aug. 11, 2014

(51) Int. Cl. A61B 18/06; A61H 39/04

(52) U.S. Cl. CPC A61B 18/06; A61H 39/04

(57) ABSTRACT

An electric-warming moxibustion device includes a casing having a long pen-like structure; an inner side of the casing being hollowed and a foremost end of the casing being opened; a battery box received in a hollowed portion of the casing; the battery box including at least one slot for receiving a battery; a circuit board received within the casing and in front of the battery box; a massage portion protruding from the front opening of the casing; the massage portion being a hollow tube with a closed front end and an opened rear end; the opening space of the massage portion being communicated to the hollow portion of the casing; an inner hollow space of the massage portion being received with a heating light; and a size of the front end of the massage portion being equal to a size of an acupuncture point of the human body.

### FIELD OF THE INVENTION

The present invention relates to moxibustion OF Chinese medicine, and in particular to an electric-warming moxibustion device.

### BACKGROUND OF THE INVENTION

Moxibustion is a treatment way in conventional Chinese medicine for increment of blood circulation, release of fatigue, etc. This kind of treatment has achieved many beneficial effects to human health. Conventionally, a moxibustion bar is used for performing moxibustion. The moxibustion bar is a hollow metal bar which has a larger head and a smaller distal end. In use, herbs are filled in the hollow space of the bar and then the herbs are burned. The performer holds the distal end of the moxibustion bar and the head portion of the bar is pointed to the acupuncture point of the patient for warming the points. The burned herbs will emit heat to warm the acupuncture points. For avoiding harming the body, a cover serves to enclose the head of the moxibustion bar.

However, this prior art treatment way has some disadvantages, such as preparing of herbs makes troubles to users. Furthermore, it is difficult to control the burning of the herbs, sometime it is overheated, and sometime it is not heat sufficiently so that the user must adjust the burning of the herbs to a desired level. The size of the hearing portion confined by the moxibustion bar (with the cover) is too large to match the size of the acupuncture points of the patients. As a result, the conventional way cannot provide better treatment effects to patients.

### SUMMARY OF THE INVENTION

Accordingly, the object of the present invention is to provide an electric-warming moxibustion device, wherein the heating source is heating lights, no herb is burned for warning the affected portion of the patients and thus it is unneeded to update the herbs again and again. It is a safe and clear way. Furthermore, the heat temperatures of the massage portion and the head cover are precisely controlled by controlling the power level of the heating light. This has overcome the disadvantages in the conventional moxibustion bar which may be over-heated or heated insufficient so to be unsuitable for warming the human body. Furthermore, the sizes and shapes of the massage portion, the head cover and the heating light are adjustable as desired. Thus the designer can design a most suitable one for precisely massaging and warming the acupuncture points of the users to have a better treating effect.

To achieve above object, the present invention provides electric-warming moxibustion device, comprising: a casing having a long pen-like structure; an inner side of the casing being hollowed and a foremost end of the casing being opened as a front opening; a battery box received in a hollowed portion of the casing; the battery box including at least one slot for receiving a battery therein; the battery box having an extending line passing through the casing and a distal end of the extending line having a plug to being connected to a socket for power transferring; a circuit board received within the casing and in front of the battery box; and a massage portion protruding from the front opening of the casing; the massage portion being a hollow tube with a closed front end and an opened rear end; the opening space of the massage portion being communicated to the hollow portion of the casing; an inner hollow space of the massage portion being received with a heating light which has a power source from the battery, a front end of the massage portion being reduced to present with a taper shape; a size of the front end of the massage portion being equal to a size of an acupuncture point of the human body.

### SUMMARY OF THE INVENTION

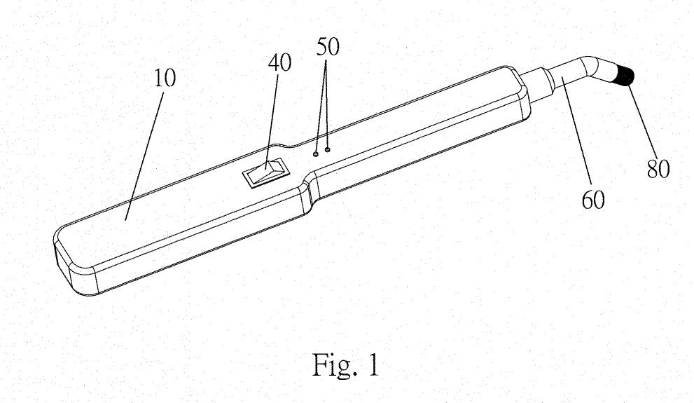

FIG. 1 is a perspective structural view of the present invention.

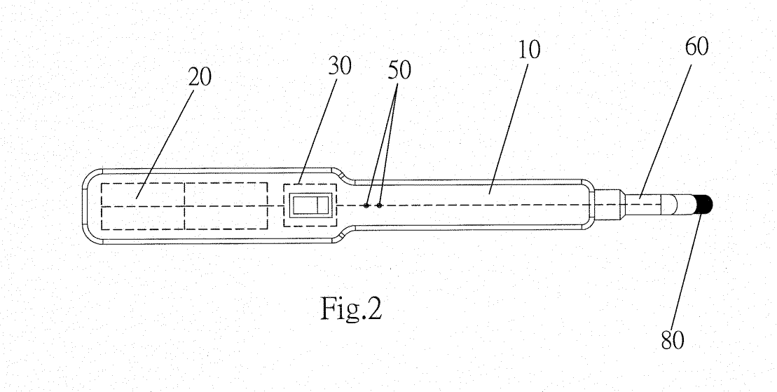

FIG. 2 is an upper view showing the structure of the present invention.

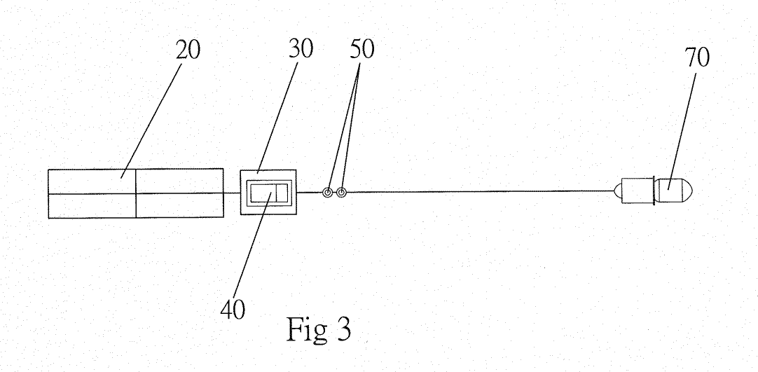

FIG. 3 shows the circuit structure of the present invention.

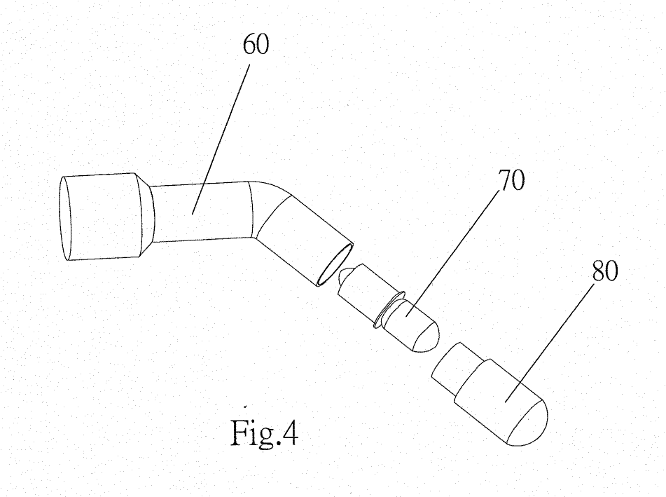

FIG. 4 is a perspective view showing the structure of the massage portion and the heating light according to the present invention.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

With reference to FIGS. 1 to 4, the electric-warming moxibustion device according to the present invention is illustrated. The device includes the following elements.

A casing 10 has a long-pen-like structure (as shown in FIGS. 1 and 2). An inner side of the casing 10 is hollowed and a foremost end of the casing 10 is opened. A rear end of the casing 10 extends to have a larger cross section for receiving other components. A front end of the casing 10 has a small cross section. The size and shape of the casing 10 is suitable to be held by human hands.

A battery box 20 is received in the enlarged rear end of the casing 10. The battery box 20 may have a plurality of slots each of which is used to receive a battery. The battery box 20 has a power line passing through the casing 10 and a distal end of the power line has a plug to being connected to a socket for power transferring.

A circuit board 30 is received within the casing 10 and is in front of the battery box 20 for locating related control means. The circuit board 30 has a power wire connected to the battery box 20 for receiving power from the battery in the battery box 20.

A switch 40 is located on the circuit board 30 (referring to FIG. 3) for switching power from the batteries in the battery box 20. An upper side of the switch 40 is formed with a press portion. In installation state, the press portion protrudes to outside of the casing 10 so that the user can control the switch 40 by pressing the press portion.

At least one LED light 50 is connected to the circuit board 30 for displaying the operation state of the present invention. Preferably, there are at least two LED lights 50, one for red light and one for yellow light for displaying different operation states.

A massage portion 60 protrudes from a front opening of the casing 10. The massage portion 60 is a hollow tube with a closed front end and an opened rear end. The opening space of the massage portion 60 is communicated to the hollow portion of the casing 10. An inner hollow space of the massage portion 60 is received with a heating light 70 which has a power source from the power wire extending from the battery box 20, while the switch 40 serves to control the power passing through the power wire so as to actuate or de-actuate the heating light 70. Preferably, a front end of the massage portion 60 is reduced to present with a taper shape. Preferably, size of the front end of the massage portion 60 is approximately equal to a size of an acupuncture point of the human body. Therefore, when the massage portion 60 touches the acupuncture point of the human body, it can properly heat the point. By adjusting the power level of the heating light 70, the temperature of the massage portion 60 can be controlled precisely.

In the present invention, the heating light 70 can be replaced by other heating element, however, heating lights are most proper ones. This is because the heat from the heating light 70 is uniformly and moderately for heating the massage portion 60 so that the heat distribution of the massage portion 60 is uniform and properly warmed.

In the present invention, the massage portion 60 may be made by metals (such as copper), plastics or ceramics, and other proper materials. Preferably, the massage portion 60 has a curved structure and the curvature thereof is matched to the requirement for patients to be treated.

A head cover 80 serves to cover the front end of the massage portion 60 so that in use, the head cover 80 is used to contact the patient's skin for warming the acupuncture points. In using, the heat from the massage portion 60 is transferred to the head cover 80 so that the head cover 80 is warmed and is able to massage of the affected portion of the patient. Preferably, in the present invention, the head cover 80 is made of cloth.

In use, the user holds the front end of the casing 10 and then actuates the switch 40 of the heating light 70. Whether the heating light 70 is opened normally is determined by the lighting up of the LED light 50. As the heating light 70 is lighted up, the head cover 80 covering the massage portion 60 will be warmly gradually. The warmness of the head cover 80 can be detected by using the hand to touch the head cover 80. If the head cover 80 has reached to properly temperature, the head cover 80 is lead to the portion of the human body (such as affected portions or portions to be treated) continuously to have the effect of heating moxibustion. The proper time period for heat is about 5 to 15 minutes.

In the present invention, the outer side of the casing 10 can be added with bar codes, RFID tags or NFC tags, and others so that the user can scan these codes or tags through notebooks, tablets, smart phones and others and then the codes or tags can be identified through network connections so as to register to a website for getting some health information about use of the present invention.

Advantages of the present invention are that the heating source is a heating light, no herb is burned for warming the affected portion of the patients and thus it is unneeded to update the herbs again and again, it being a safe and clear way. Furthermore, the heat temperatures of the massage portion and the head cover are precisely controlled by controlling the power level of the heating light, the disadvantages in the conventional moxibustion bar which may be over-heated or over-cooled being overcome. Furthermore, the sizes and shapes of the massage portion, the head cover 80 and the heating light are adjustable as desired. As a result, designers can design a most suitable one for precisely massaging and warming the acupuncture points of the users to have a better treating effect.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An electric-warming moxibustion device comprising:
a casing having a long pen-like structure; an inner side of the casing being hollowed and a foremost end of the casing being opened as a front opening;
a battery box received in a hollowed portion of the casing; the battery box including at least one slot for receiving a battery therein;
a circuit board received within the casing and in front of the battery box;
a massage portion protruding from the front opening of the casing; the massage portion being a hollow tube with a closed front end and an opened rear end; the opening space of the massage portion being communicated to the hollow portion of the casing; an inner hollow space of the massage portion being received with a heating light or bulb which has a power source from the battery, a front end of the massage portion being reduced to present with a taper shape; and a size of the front end of the massage portion being equal to a size of an acupuncture point of the human body.

2. The electric-warming moxibustion device as claimed in claim 1, wherein a rear end of the casing extends to have a larger cross section for receiving other components; and a front end of the casing has a small cross section for extending the whole length; and a size and a shape of the casing is suitable to be held by human hands.

3. The electric-warming moxibustion device as claimed in claim 1, further comprising: at least one LED light is connected to the circuit board for displaying the operation state. 4, The electric-warming moxibustion device as claimed in claim 1, wherein the massage portion is made of metals, plastics or ceramics.

5. The electric-warming moxibustion device as claimed in claim 1, wherein the massage portion has a curved structure and the curvature thereof is matched to the requirement for patents to be treated.

6. The electric-warming moxibustion device as claimed in claim 1, further comprising a head cover for covering the front end of the massage portion so that in use, the head cover is used to contact the patient's skin for warming the acupuncture points.

7. The electric-warming moxibustion device as claimed in claim 1, wherein an outer side of the casing is printed with bar codes, RFID tags or NFC tags so that the user can scan these codes or tags through notebooks, tablets, smart phones and others and then the codes or tags can be identified through network connections so as to register to a website for getting some health information about use of the present invention.

9. The electric-warming moxibustion device as claimed in claim 1, further comprising a switch located on the circuit board for switching power from the batteries in the battery box; an upper side of the switch is formed with a press portion; in installation state, the press portion protrudes to outside of the casing.

10. The electric-warming moxibustion device as claimed in claim 1, wherein the battery box has a power wire passing through the casing and a distal end of the power wire having a plug to be connected to a socket for power transferring;
